(19) United States
(12) Patent Application Publication
(10) Pub. No.: US 2014/0100403 A1
(43) Pub. Date: Apr. 10, 2014

(54) LOW PRESSURE TRANSALKYLATION PROCESS

(71) Applicant: UOP LLC, Des Plaines, IL (US)

(72) Inventors: Naiyl A. Rashid, Carpentersville, IL (US); Edwin P. Boldingh, Arlington Heights, IL (US); Raymond Shih, Elgin, IL (US); Marc R. Schreier, Romeoville, IL (US); David S. Lafyatis, Schaumburg, IL (US)

(73) Assignee: UOP LLC, Des Plaines, IL (US)

(21) Appl. No.: 13/645,998

(22) Filed: Oct. 5, 2012

(51) Int. Cl. C07C 5/25; C07C 2/54

(52) U.S. Cl. 585/475

(57) ABSTRACT

A process for transalkylation is described. The process operates at a lower pressure than a typical transalkylation processes, and provides higher benzene purity with comparable or lower ring loss compared to the typical transalkylation process. The xylene selectivity is comparable to or higher than the standard process, and the ethyl benzene selectivity is comparable to or lower than the standard process.

### FIELD OF THE INVENTION

The present invention relates to a process for the conversion of aromatic hydrocarbons, and more specifically to a low pressure process for the transalkylation of aromatic hydrocarbons to obtain xylenes.

### BACKGROUND OF THE INVENTION

Xylene isomers are produced in large volumes from petroleum as feedstocks for a variety of important industrial chemicals. The most important of the xylene isomers is para-xylene, the principal feedstock for polyester which continues to enjoy a high growth rate from a large base demand. Ortho-xylene is used to produce phthalic anhydride, which has high-volume but mature markets. Meta-xylene is used in lesser but growing volumes for such products as plasticizers, azo dyes and wood preservers. Ethylbenzene is generally present in xylene mixtures and is occasionally recovered for styrene production, but usually is considered a less-desirable component of C₈ aromatics.

Among the aromatic hydrocarbons, the overall importance of the xylenes rivals that of benzene as a feedstock for industrial chemicals. Neither the xylenes nor benzene is produced from petroleum by the reforming of naphtha in sufficient volume to meet demand. As a result, conversion of other hydrocarbons is necessary to increase the yield of xylenes and benzene. Toluene is commonly dealkylated to produce benzene or disproportionated to yield benzene and C₈ aromatics from which the xylene isomers are recovered. More recently, processes have been commercialized to transalkylate heavier aromatics along with toluene selectively to increase the yield xylenes from aromatics complexes.

The art teaches a variety of catalysts for the transalkylation of aromatic hydrocarbons. A wide range of zeolites, including mordenite, have been disclosed as effective transalkylation catalysts. Shaped catalysts, multiple zeolites, metal modifiers, and treatments such as steam calcination have been described as increasing the effectiveness of the catalysts.

There is a need to improve processes for the conversion of heavy hydrocarbons.

### SUMMARY OF THE INVENTION

One aspect of the invention is a process for transalkylation. In one embodiment, the process includes contacting a feedstream comprising one or more of C₇, C₉, C₁₀, and C₁₁₊ aromatics with a transalkylation catalyst which comprises (1) an aggregate zeolitic material comprising globular aggregates of crystallites having a MOR framework type comprising 12-ring channels, a mesopore volume of at least about 0.10 cc/gram, a mean crystallite length parallel to the direction of the 12-ring channels of about 60 nm or less, the number of 12-ring channel-openings per gram of zeolite of at least 1×10¹⁹, and a silica-alumina (Si/Al₂) mole ratio of from about 8 to about 50, (2) a binder comprising one or more of alumina, silica, silica-alumina, aluminum phosphate, and (3) a metal component selected from the group consisting of groups VIB(6), VIIB(7), VIII(8-10) and IVA(14) of the Periodic Table, and mixtures thereof under first transalkylation conditions at a pressure of about 2.1 MPa (300 psi) or less, to obtain a product stream having an increased concentration of C₈ aromatics relative to the feedstream, a benzene purity higher than a benzene purity under the first transalkylation conditions at a pressure of about 2.8 MPa (400 psi), a ring loss comparable to or lower than a ring loss under the first transalkylation conditions at the pressure of about 2.8 MPa (400 psi), a xylene selectivity comparable to or higher than a xylene selectivity under the first transalkylation conditions at the pressure of about 2.8 MPa (400 psi), and an ethyl benzene selectivity comparable to or lower than an ethyl benzene selectivity under the first transalkylation conditions at the pressure of about 2.8 MPa (400 psi).

### BRIEF DESCRIPTION OF THE DRAWINGS

FIG. 1A is a graph comparing benzene impurities in a transalkylation process at 2.8 MPa (400 psi) and 1.7 MPa (250 psi).

FIG. 1B is a graph comparing calculated benzene impurities after fractionation in a transalkylation process at 2.8 MPa (400 psi) and 1.7 MPa (250 psi).

FIG. 2 is a graph comparing ring loss in a transalkylation process at 2.8 1MPa (400 psi) and 1.7 MPa (250 psi).

FIG. 3 is a graph comparing xylene selectivity in a transalkylation process at 2.8 MPa (400 psi) and 1.7 MPa (250 psi).

FIG. 4A is a graph comparing benzene impurities in a transalkylation process at 2.8 MPa (400 psi) and 1.7 MPa (250 psi) for various feed compositions.

FIG. 4B is a graph comparing calculated benzene impurities after fractionation in a transalkylation process at 2.8 MPa (400 psi) and 1.7 MPa (250 psi) for various feed compositions.

FIG. 5 is a graph comparing ring loss in a transalkylation process at 2.8 MPa (400 psi) and 1.7 MPa (250 psi) for various feed compositions.

FIG. 6 is a graph comparing xylene selectivity in a transalkylation process at 2.8 MPa (400 psi) and 1.7 MPa (250 psi) for various feed compositions.

FIG. 7 is a graph comparing ethyl benzene selectivity in a transalkylation process at 2.8 MPa (400 psi) and 1.7 MPa (250 psi) for various feed compositions.

FIG. 8 is a graph comparing ethyl benzene selectivity in a transalkylation process at 1.7 MPa (250 psi) and 1.2 MPa (175 psi).

### DETAILED DESCRIPTION OF THE INVENTION

A lower pressure transalkylation process which produces higher benzene purity with comparable or lower ring loss, comparable or higher xylene selectivity, and comparable or lower ethyl benzene selectivity is provided. “Ring loss” means the molar loss of aromatic rings across the transalkylation process. The lower pressure transalkylation process reduces equipment costs for new installations. In addition, existing low pressure units can be converted from other processes without having to upgrade to higher pressure reactors.

A catalyst containing UZM-14 zeolite and a process for transalkylation and/or disproportionation of heavy hydrocarbons, such as C₇, C₉, C₁₀, and C₁₁₊ aromatics, to obtain a high yield of xylenes are described, for example, in U.S. Pat. Nos. 7,605,295, 7,626,064, and 7,687,423, each of which is incorporated herein by reference.

In this process, the feed to the transalkylation reaction zone (which is described more fully below) is usually first heated by indirect heat exchange against the effluent of the reaction zone and is then heated to reaction temperature by exchange with a warmer stream, steam or a furnace. The feed is passed through a reaction zone, which may comprise one or more individual reactors. Passage of the combined feed through the reaction zone produces an effluent stream comprising unconverted feed and product hydrocarbons. This effluent is normally cooled by indirect heat exchange against the stream entering the reaction zone and then further cooled through the use of air or cooling water. The effluent may be passed to a stripping column in which substantially all C5 and lighter hydrocarbons present in the effluent are concentrated into an overhead stream and removed from the process. An aromatics-rich stream is recovered as net stripper bottoms, which is referred to as the transalkylation effluent.

The transalkylation or disproportionation reaction can be effected in contact with the catalytic composite in any conventional or otherwise convenient manner and may comprise a batch or continuous type of operation, with a continuous operation being preferred. The transalkylation catalyst is usefully disposed as a fixed bed in a reaction zone of a vertical tubular reactor with the alkylaromatic feed stock charged through the bed in an upflow or downflow manner. Conditions employed in the transalkylation zone normally include a temperature of from about 200° C. to about 540° C., or between about 200° C. to about 480° C. The transalkylation zone is operated at moderately elevated pressures broadly ranging from about 100 kPa to about 6 MPa absolute. The transalkylation reaction can be effected over a wide range of space velocities, i.e., volume of charge per volume of catalyst per hour; weight hourly space velocity (WHSV) generally is in the range of from about 1 to about 7 hr⁻¹. The catalyst is particularly noteworthy for its relatively high stability at a high activity level.

The transalkylation zone effluent may be further separated in a distillation zone comprising at least one distillation column to produce a benzene product stream. Various flow schemes and combinations of distillation columns to separate transalkylation zone effluent via fractional distillation are well known in the art. In addition to the benzene product stream, the distillation zone may produce a toluene product stream and a C₈₊ product stream. See, e.g., U.S. Pat. No. 7,605,295. It is also known that the transalkylation zone stripper column may be designed and operated to produce a benzene product stream. See, e.g., U.S. Pat. No. 6,740,788. Thus, the reaction product stream contains a benzene fraction that may be separated by fractional distillation to produce a benzene product stream. A benzene product of acceptable purity according to the present invention is benzene which generally would meet specifications for further chemical processing merely by fractional distillation of the reaction products, preferably, without limitation, having a purity of at least about 99.86% by weight.

In another embodiment, the transalkylation effluent is separated into a light recycle stream, a mixed C8 aromatics product and a heavy-aromatics stream. The mixed C8 aromatics product can be sent for recovery of para-xylene and other valuable isomers. The light recycle stream may be diverted to other uses such as to benzene and toluene recovery, but alternatively is recycled partially to the transalkylation zone. The heavy recycle stream contains substantially all of the C9 and heavier aromatics and may be partially or totally recycled to the transalkylation reaction zone.

In the process described above, the typical operating conditions include a temperature of about 350° C., a pressure of about 2.8 MPa absolute (about 400 psia), and a WHSV of about 2 to about 4 hr⁻¹. However, under these conditions, the benzene purity may be unacceptably low (i.e., less than 99.8% after fractionation) because the process causes saturation of some of the aromatic rings, leading to some impurities with boiling points close to that of benzene that cannot efficiently be separated from the benzene by fractionation. This is partially due to the very high activity and thus low operating temperatures of the UZM-14 containing catalyst.

The pressure was reduced in order to improve the benzene purity while maintaining the same feedstream composition and transalkylation conditions such as WHSV, H₂:HC, and overall feedstream conversion (i.e., conversion of C₇, C₉, C₁₀, and C₁₁₊ aromatics). Overall feedstream conversion can be calculated using the following equations:

$$MassBasisIn ( g \text{/} hr ) = {\sum }_{i} {MassCompIn}_{i} ( g \text{/} hr )$$

$$MassBasisOut ( g \text{/} hr ) = {\sum }_{i} {MassCompOut}_{i} ( g \text{/} hr )$$

$$Conversion ( mass \% ) = ( 1 - \frac{MassBasisOut}{MassBasisIn} ) \cdot 100$$

where “i” is hydrocarbons such as toluene, propyl benzene, methyl ethyl benzene, trimethyl benzene, indane, methyl propyl benzene, diethyl benzene, dimethyl ethyl benzene, and/or tetra methyl benzene and the like.

The pressure was generally about 2.1 MPa absolute (about 300 psia) or less, or about 1.7 MPa absolute (about 250 psia) or less. The temperature was increased about 20° C. to maintain the conversion.

Lowering the pressure improved the benzene purity. The benzene purity after fractionation was generally at least about 99.90 wt %, or at least about 99.95 wt %. “Benzene purity” means either the benzene purity measured after the transalkylation process or the benzene purity after fractionation. “Benzene purity after fractionation” means the benzene purity after fractionation either measured or calculated from the benzene purity after the transalkylation process.

Normally, lowering the pressure requires a higher operating temperature to maintain the desired conversion, which typically results in higher ring loss and lower xylene selectivity. In addition, lowering the pressure normally results in faster deactivation of the catalyst, resulting in a shorter catalyst life, often an unacceptably shorter life.

However, with the UZM-14 containing catalyst, the ring loss unexpectedly did not increase at the lower pressure. Rather, it was comparable to, or lower than, the ring loss at the higher pressure under the same transalkylation conditions. By “comparable to” the ring loss at higher pressure is meant that the ring loss is within ±5% of the ring loss at the higher pressure. The ring loss is generally at least about 10% lower than the ring loss at the higher pressure, or at least about 15% lower, or at least about 20% lower, or at least about 25% lower. The ring loss is generally less than about 1.5 mol %, or less than about 1.2 mol %, or less than about 0.9 mol %.

In addition, the xylene selectivity at the lower pressure was comparable to or higher than the xylene selectivity at the higher pressure under the same transalkylation conditions. By “comparable to” the xylene selectivity is meant that the xylene selectivity is within ±0.2% of the xylene selectivity at the higher pressure.

Ethyl benzene is considered the least desirable of the C8 aromatic compounds. The ethyl benzene selectivity at the lower pressure was comparable to or lower than the ethyl benzene selectivity at the higher pressure under the same transalkylation conditions. By “comparable to” the ethyl benzene selectivity is meant that the ethyl benzene is within ±5% of the ethyl benzene selectivity at the higher pressure. The ethyl benzene is generally at least about 10% lower than the ethyl benzene selectivity at the higher pressure, or at least about 15% lower, or at least about 20% lower, or at least about 25% lower, or at least about 30% lower, or at least about 35% lower, or at least about 40% lower.

The catalyst life is expected to be at least about 4 years, or at least about 5 years, or at least about 6 years.

The aromatics-rich feed stream to the transalkylation or disproportionation process may be derived from a variety of sources, including without limitation, catalytic reforming, pyrolysis of naphtha, distillates or other hydrocarbons to yield light olefins and heavier aromatics-rich byproducts, and catalytic or thermal cracking of heavy oils to yield products in the gasoline range. Products from pyrolysis or other cracking operations generally will be hydrotreated according to processes well known in the industry before being charged to the complex in order to remove sulfur, olefins and other compounds which would affect product quality. Light cycle oil also may be beneficially hydrocracked to yield lighter components which can be reformed catalytically to yield the aromatics-rich feed stream. If the feed stream is catalytic reformate, the reformer preferably is operated at high severity for high aromatics yield with a low concentration of nonaromatics in the product. The reformate also advantageously is subjected to olefin saturation to remove potential product contaminants and materials that could polymerize to heavy nonconvertibles in a transalkylation process. Such processing steps are described in U.S. Pat. No. 6,740,788 B 1, which is incorporated herein by reference.

The feed stream to a transalkylation or disproportionation process can be a substantially pure alkylaromatic hydrocarbon of from about 6 to about 15 carbon atoms, a mixture of such alkylaromatic hydrocarbons, or a hydrocarbon fraction rich in said alkylaromatics. The feedstream comprises alkylaromatic hydrocarbons of the general formula C6H₍₆₋ₙ₎Rₙ, where n is an integer from 0 to 5 and R is CH₃, C₂H₅, C₃H₇, or C₄H₉, in any combination. Suitable alkylaromatic hydrocarbons include, but are not limited to, benzene, toluene, ortho-xylene, meta-xylene, para-xylene, ethylbenzene, ethyltoluenes, propylbenzenes, tetramethylbenzenes, ethyl-dimethylbenzenes, diethylbenzenes, methylpropylbenzenes, ethylpropylbenzenes, triethylbenzenes, diisopropylbenzenes, and mixtures thereof. The feed stream also may contain lesser concentrations of nonaromatics such as pentanes, hexanes, heptanes and heavier paraffins along with methylcyclopentane, cyclohexane and heavier naphthenes. Pentanes and lighter paraffins generally will have been removed before processing in the aromatics complex. The combined transalkylation feed preferably contains no more than about 10 wt-% nonaromatics. Olefins preferably are restricted to a Bromine Index of no more than about 1000, and preferably no more than about 500.

A preferred component of the feedstock is a heavy-aromatics stream comprising C9 aromatics, thereby effecting transalkylation of toluene and C9 aromatics to yield additional xylenes. Benzene may also be transalkylated to yield additional toluene. Indane may be present in the heavy-aromatics stream, although it is not a desirable component to effect high yields of C8 aromatics product. C10+ aromatics also may be present, preferably in an amount of 30% or less of the feed. The heavy-aromatics stream preferably comprises at least about 90 mass-% aromatics, and may be derived from the same or different known refinery and petrochemical processes as the benzene and toluene feedstock and/or may be recycled from the separation of the product from transalkylation.

The UZM-14 zeolite has an empirical composition in the as-synthesized form on an anhydrous basis expressed by the empirical formula:

Mₘⁿ⁺Rᵣ^{p-}Al₁₋ₓSiO_{z}

where M is at least one exchangeable cation and is selected from the group consisting of alkali and alkaline earth metals including but not limited to lithium, sodium, potassium, rubidium, cesium, calcium, strontium, barium and mixtures thereof. R is at least one organic cation selected from the group consisting of protonated amines, protonated diamines, quaternary ammonium ions, diquaternary ammonium ions, protonated alkanolamines, and quaternized alkanolammonium ions. Relating the components, “m” is the mole ratio of M to Al and varies from about 0.05 to about 0.95 “r” is the mole ratio of R to Al and has a value of about 0.05 to about 0.95, “n” is the weighted average valence of M and has a value of about 1 to about 2, “p” is the weighted average valence of R and has a value of about 1 to about 2, “y” is the mole ratio of Si to Al and varies from about 3 to about 50 and “z” is the mole ratio of 0 to Al and has a value determined by the equation:

z=(mn+rp+3+4y)/2

The UZM-14 containing catalyst comprises a refractory inorganic-oxide binder and a metal component. The inorganic-oxide binder component of the invention comprises such materials as alumina, silica, zirconia, titania, thoria, boria, magnesia, chromia, stannic oxide, and the like as well as combinations and composites thereof, for example alumina-silica, alumina-zirconia, alumina-titania, aluminum phosphate, and the like. The binder preferably is selected from one or more of alumina, silica and silica-alumina. Alumina is an especially preferred refractory inorganic oxide for use herein, particularly with respect to the manufacture of a catalytic composite for use in the transalkylation of alkylaromatic hydrocarbons. The alumina may be any of the various hydrous aluminum oxides or alumina gels such as alpha-alumina monohydrate of the boehmite structure, alpha-alumina trihydrate of the gibbsite structure, beta-alumina trihydrate of the bayerite structure, and the like, the first mentioned alpha-alumina monohydrate being preferred. An alternative preferred binder is aluminum phosphate as described in U.S. Pat. No. 4,629,717, which is incorporated herein by reference.

The UZM-14 containing catalyst optionally may comprise an additional zeolitic component, The additional zeolite component preferably is selected from one or more of MFI, MEL, EUO, FER, MFS, MOR, MTT, MTW, MWW, MAZ, TON and FAU (IUPAC Commission on Zeolite Nomenclature) and UZM-8 (see WO 2005/113439, incorporated herein by reference thereto). More preferably, particularly when the catalyst is used in a transalkylation process, the additional zeolitic component consists essentially of MFI. Suitable total zeolite amounts in the catalyst range from about 1 to about 100 wt-%, preferably from about 10 to about 95 wt-%, and more preferably between about 60 and about 90 wt-%.

The catalyst preferably comprises a metal component comprising one or more elements selected from groups VIB(6), VIIB(7), VIII(8-10), IB(11), IIB(12), IIIA(13) and IVA(14) of the Periodic Table. Preferably the metal component is selected from one or more of rhenium, nickel, cobalt, molybdenum and tungsten when the catalyst is used in a transalkylation process. The catalyst also may contain phosphorus. Suitable metal amounts in the transalkylation catalyst range from about 0.01 to about 15 wt-% on an elemental basis, with the range from about 0.1 to about 12 wt-% being preferred, and the range from about 0.1 to about 10 wt-% being highly preferred. The catalyst also preferably has been subjected to a presulfiding step to incorporate from about 0.05 to about 2 wt.-% sulfur on an elemental basis. This presulfiding step may take place either during the manufacture of the catalyst or after the catalyst has been loaded into a process unit.

### EXAMPLE 1

The performance of the UZM-14 catalyst in a transalkylation process at a pressure of 2.8 MPa (400 psi) was compared to that at a pressure of 1.7 MPa (250 psi). The feed was 50% toluene and 50% A9+ aromatics. The ratio of H₂:HC was 3.0, the weight hourly space velocity (WHSV) was 3.0, and the overall conversion was 50% for both runs.

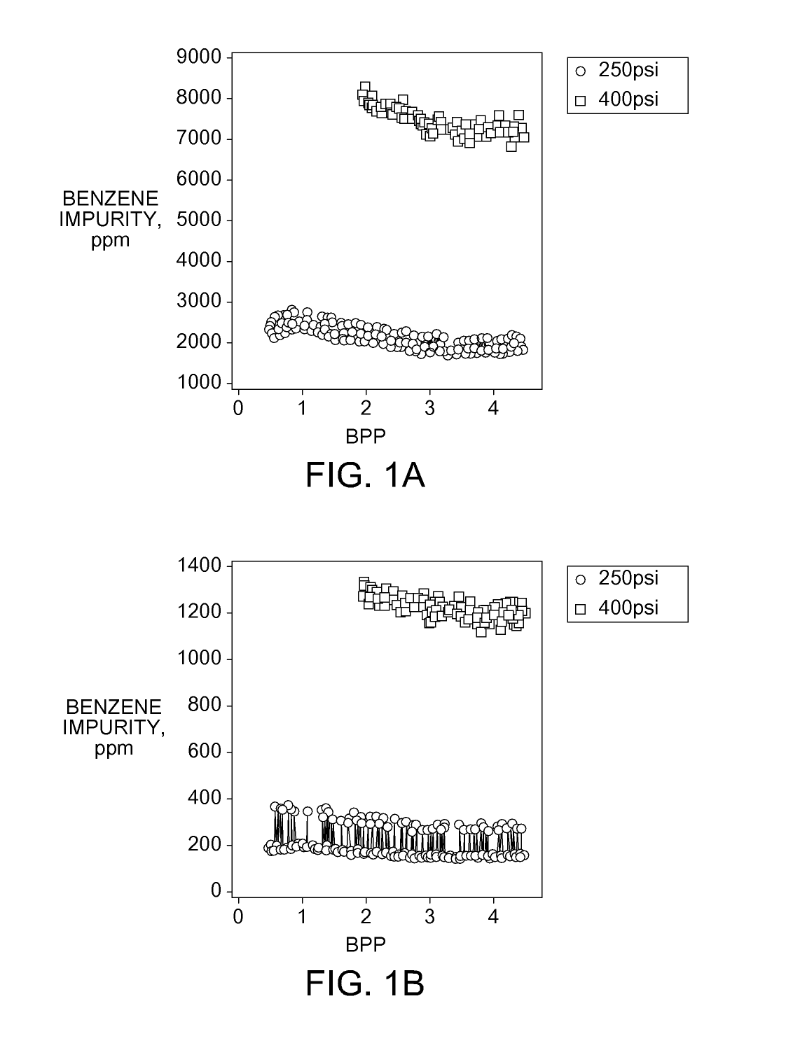

FIG. 1A shows the measured level of benzene impurities after transalkylation, and FIG. 1B shows the level of benzene impurities following fractionation calculated from the measured level of FIG. 1A by simulating a stripper column and a distillation column. “BPP” indicates barrels of feed processed per pound of catalyst, and thus the period in the operating cycle, “0” being the start of the cycle. As shown in FIG. 1B, at 2.8 MPa (400 psi), the benzene purity after fractionation was 99.88%, which does not meet the required level of 99.90%. In contrast, the benzene purity after fractionation at 1.7 MPa (250 psi) was 99.97%. The scatter in the graph of benzene impurity after fractionation is due to the fact that the level of some of the benzene coboilers is at or below the detection limit of the gas chromatograph. As a result, they appear in the data sometimes, while they do not appear at other times.

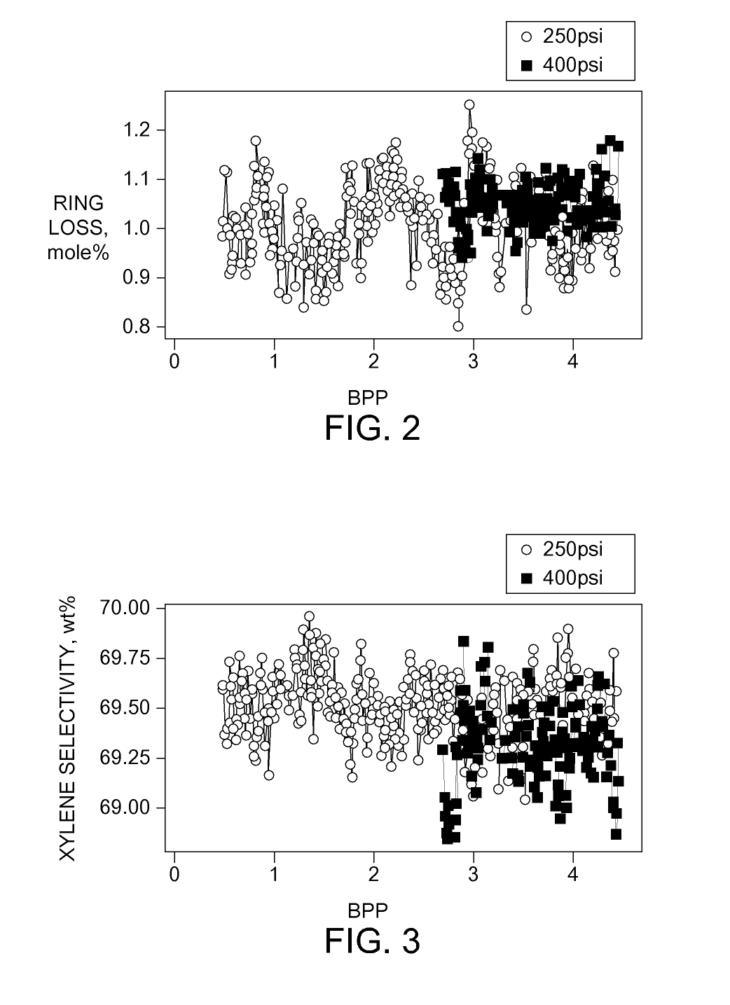

FIG. 2 shows an average ring loss of 1.08 mol % at 2.8 MPa (400 psi) and 1.07 mol % at 1.7 MPa (250 psi). The xylene selectivity was 69.7% at 2.8 MPa (400 psi) and 69.8% at 1.7 MPa (250 psi) as shown in FIG. 3.

Thus improved benzene purity with no loss of desired product selectivity is demonstrated at reduced pressure conditions.

### EXAMPLE 2

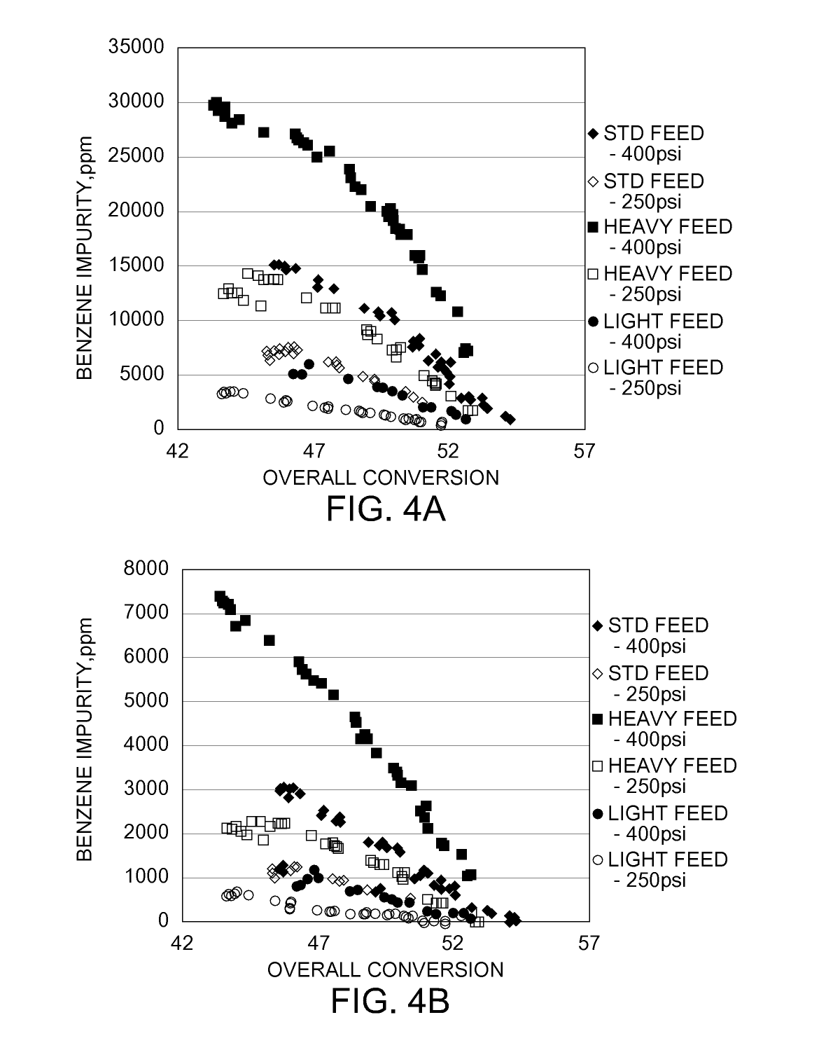

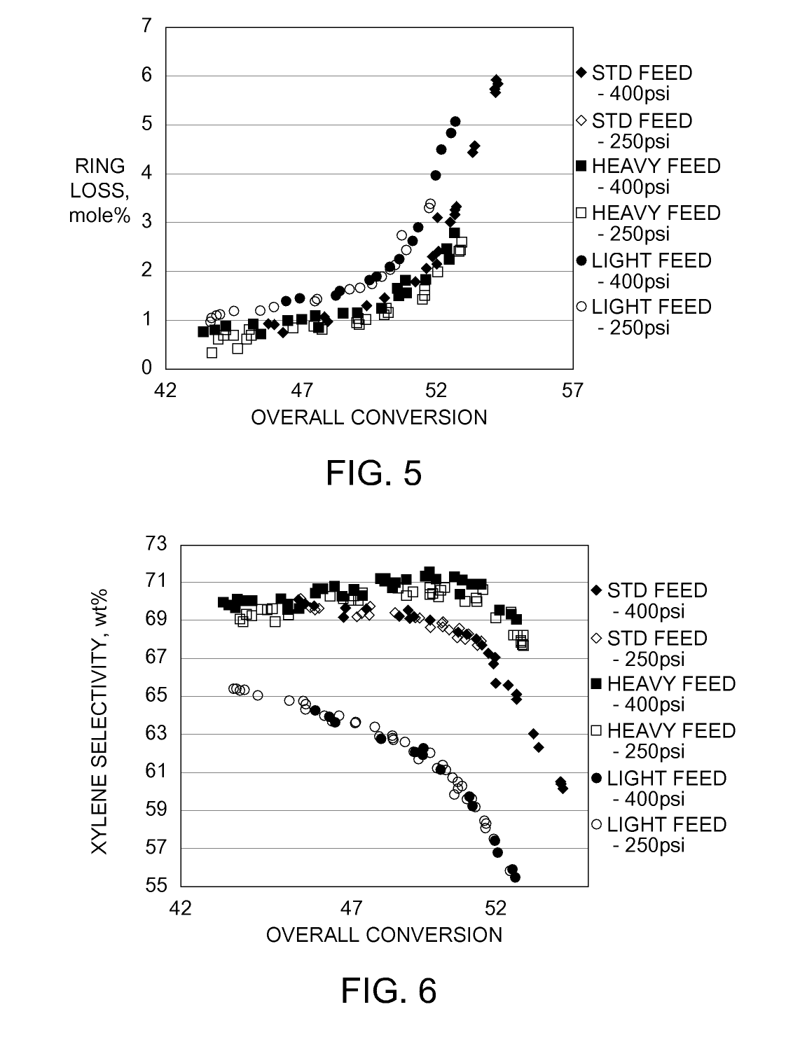

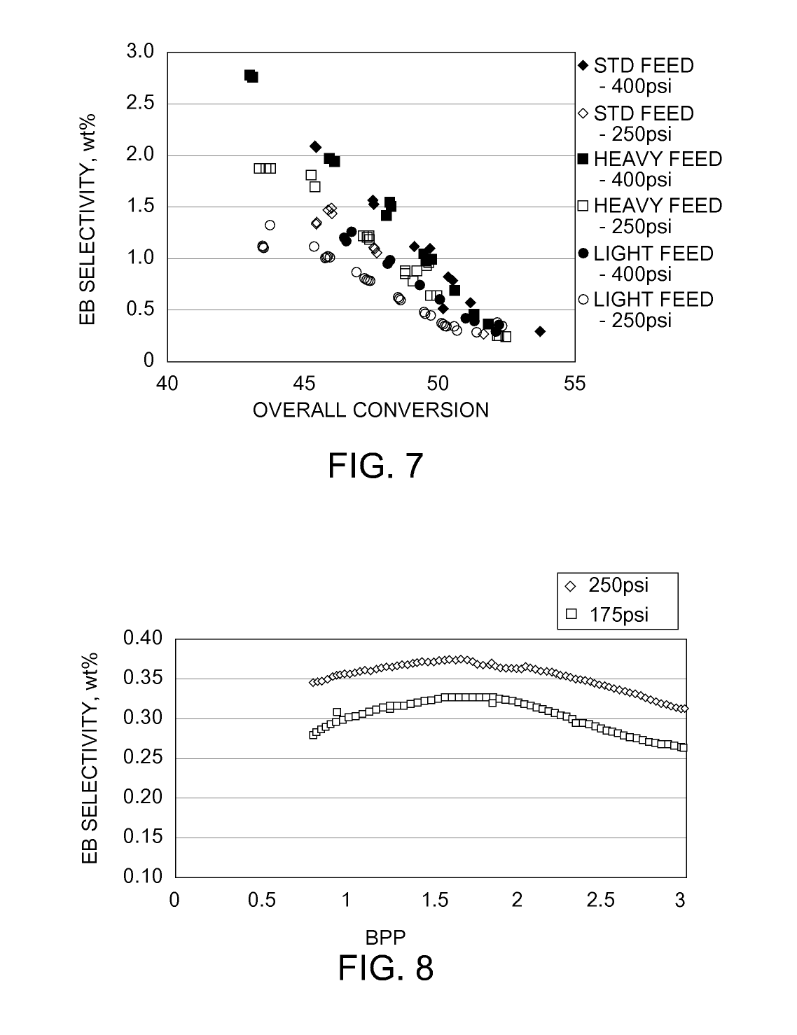

Three feed compositions (25% toluene/75% A9+ aromatics (heavy feed), 50% toluene/50% A9+ aromatics (standard feed), and 75% toluene/25% A9+ aromatics (light feed)) were tested at 1.7 MPa (250 psi) to ensure that lowering the pressure did not have a negative effect on the catalyst stability and performance. As shown in FIGS. 4A-7, operating at 1.2 MPa (175 psi) showed improved benzene purity and comparable ring loss, and xylene selectivity, and comparable or lower ethyl benzene selectivity compared to the process run at 2.8 MPa (400 psi) for all three feed compositions over a wide range of commercially relevant conversion levels.

Tests at 1.2 MPa (175 psi) with a feed of 75% toluene and 25% A9+ aromatics, a ratio of H₂:HC of 3.0, a weight hourly space velocity (WHSV) of 3.0, and an overall conversion of 50% showed improved benzene purity, lower ethyl benzene selectivity, and comparable ring loss, and xylene selectivity compared to the process run at 2.8 MPa (400 psi). FIG. 8 shows the ethyl benzene selectivity at 1.2 MPa (175 psi) compared to 1.7 MPa (250 psi).

Table 1 compares the deactivation rate of a commercially available catalyst from UOP LLC in the transalkylation process at 2.8 MPa (400 psi) with a UZM-14 containing catalyst at a pressure of 2.8 MPa (400 psi) and 1.7 MPa (250 psi). The rate of deactivation is the temperature rise required over a given amount of time usage to maintain the conversion over the catalyst. The commercial catalyst has a demonstrated life of at least 4 years. Because the rate of deactivation for the UZM-14 containing catalyst at 1.7 MPa (250 psi) is lower than that of the commercial catalyst, it is expected that the UZM-14 containing catalyst will have a life of at least 4 years at 1.7 MPa (250 psi).

TABLE 1

| | | Relative rate of |
|---|---|---|
| Catalyst | Pressure | Deactivation |
| Commercial Catalyst | 400 psi | 1.0 |
| UZM-14 Containing | 400 psi | 0.11 |
| Catalyst | | |
| UZM-14 Containing | 250 psi | 0.34 |
| Catalyst | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for transalkylation comprising:
contacting a feedstream comprising one or more of C₇, C₉, C₁₀, and C₁₁₊ aromatics with a transalkylation catalyst which comprises (1) an aggregate zeolitic material comprising globular aggregates of crystallites having a MOR framework type comprising 12-ring channels, a mesopore volume of at least about 0.10 cc/gram, a mean crystallite length parallel to the direction of the 12-ring channels of about 60 nm or less, the number of 12-ring channel-openings per gram of zeolite of at least 1×10¹⁹, and a silica-alumina (Si/Al₂) mole ratio of from about 8 to about 50, (2) a binder comprising one or more of alumina, silica, silica-alumina, aluminum phosphate, and (3) a metal component selected from the group consisting of groups VIB(6), VIIB(7), VIII(8-10) and IVA(14) of the Periodic Table, and mixtures thereof under first transalkylation conditions at a pressure of about 2.1 MPa (300 psi) or less, to obtain a product stream having an increased concentration of C₈ aromatics relative to the feedstream, a benzene purity higher than a benzene purity under the first transalkylation conditions at a pressure of about 2.8 MPa (400 psi), a ring loss comparable to or lower than a ring loss under the first transalkylation conditions at the pressure of about 2.8 MPa (400 psi), a xylene selectivity comparable to or higher than a xylene selectivity under the first transalkylation conditions at the pressure of about 2.8 MPa (400 psi), and an ethyl benzene selectivity comparable to or lower than an ethyl benzene selectivity under the first transalkylation conditions at the pressure of about 2.8 MPa (400 psi).

2. The process of claim 1 wherein the benzene purity is at least 99.90 wt % after fractionation.

3. The process of claim 1 wherein the benzene purity is at least 99.95 wt % after fractionation.

4. The process of claim 1 wherein the ring loss is less than about 1.5 mol %.

5. The process of claim 1 wherein the ring loss is less than about 1.2 mol %.

6. The process of claim 1 wherein the ring loss is less than about 0.9 mol %.

7. The process of claim 1 wherein the pressure is about 1.7 MPa (250 psi) or less.

8. The process of claim 1 wherein the catalyst life is at least about 4 years.

9. The process of claim 1 wherein an overall feedstream conversion is at least about 40%.

10. The process of claim 1 wherein an overall feedstream conversion is at least about 45%.

11. The process of claim 1 wherein the first transalkylation conditions include a WHSV of about 2 to about 4, and a ratio of H₂:HC of about 3 to about 4.

12. The process of claim 1 wherein the feedstream comprises about 40 wt % to about 75 wt % C₇ aromatics and about 25 wt % to about 60 wt % C₉₊ aromatics

13. A process for transalkylation comprising:
contacting a feedstream comprising one or more of C₇, C₉, C₁₀, and C₁₁₊ aromatics with a transalkylation catalyst which comprises (1) an aggregate zeolitic material comprising globular aggregates of crystallites having a MOR framework type comprising 12-ring channels, a mesopore volume of at least about 0.10 cc/gram, a mean crystallite length parallel to the direction of the 12-ring channels of about 60 nm or less, the number of 12-ring channel-openings per gram of zeolite of at least 1×10¹⁹, and a silica-alumina (Si/Al₂) mole ratio of from about 8 to about 50, (2) a binder comprising one or more of alumina, silica, silica-alumina, aluminum phosphate, and (3) a metal component selected from the group consisting of groups VIB(6), VIIB(7), VIII(8-10) and IVA(14) of the Periodic Table, and mixtures thereof under first transalkylation conditions at a pressure of about 2.1 MPa (300 psi) or less, to obtain a product stream having an increased concentration of C₈ aromatics relative to the feedstream, a benzene purity of at least 99.90 wt % after fractionation, a ring loss less than 1.5 mol %, a xylene selectivity comparable to or higher than a xylene selectivity under the first transalkylation conditions at the pressure of about 2.8 MPa (400 psi), an ethyl benzene selectivity comparable to or lower than an ethyl benzene selectivity under the first transalkylation conditions at the pressure of about 2.8 MPa (400 psi), and wherein the catalyst has a life of at least about 4 years.

14. The process of claim 13 wherein the ring loss is less than 1.2 mol %.

15. The process of claim 13 wherein the pressure is about 1.7 MPa (250 psi) or less.

16. The process of claim 13 wherein the benzene purity is at least 99.95 wt % after fractionation.

17. The process of claim 13 wherein an overall feedstream conversion is at least about 40%.

18. The process of claim 13 wherein an overall feedstream conversion is at least about 45%.

19. The process of claim 13 wherein the first transalkylation conditions include a WHSV of about 2 to about 4, a ratio of H₂:HC of about 3 to about 4.

20. The process of claim 13 wherein the feedstream comprises about 40 wt % to about 75 wt % C₇ aromatics and about 25 wt % to about 60 wt % C₉₊ aromatics.
